(19) United States
(12) Patent Application Publication
(10) Pub. No.: US 2003/0199938 A1
(43) Pub. Date: Oct. 23, 2003

(54) Precise cardiac lead placement based on impedance measurements

(76) Inventors: Karel Smits, Munslergeleen (NL); Chester Struble, Eijsden (NL)

Correspondence Address: MEDTRONIC, INC., 710 MEDTRONIC PARKWAY NE, MS-LC340, MINNEAPOLIS, MN, 55432-5604, US

(21) Appl. No.: 10/127,034

(22) Filed: Apr. 22, 2002

(51) Int. Cl. A61N 1/36; A61N 1/37

(52) U.S. Cl. 607/27

(57) ABSTRACT

A method of implanting a medical lead includes measuring impedance associated with an electrode disposed on an implantable lead at a cardiac location, and positioning the electrode based on the measured impedance. The method may be used specifically for positioning a medical lead within the interventricular septum that separates the left and right ventricles of the heart. By using impedance as an indication of the positioning of the lead, the implantation process can be improved and simplified, and negative effects associated with piercing the left ventricular wall can be avoided.

### FIELD OF THE INVENTION

The invention relates to implantable medical devices, and more particularly, to cardiac pacemakers.

### BACKGROUND

The technology of cardiac pacemakers has developed in sophistication and functionality over the years. In general, cardiac pacemakers are designed to control the heart by correcting or compensating for various heart abnormalities which can be encountered in human or mammalian patients. For example, cardiac pacemakers may provide therapeutic stimulation to the heart by delivering therapeutic pulses such as pacing, cardioversion or defibrillation pulses.

The pulses can be delivered to the heart via electrodes disposed on implantable cardiac leads. For example, the leads may position the electrodes with respect to one or more cardiac locations so that the pacemaker can deliver pulses to the appropriate locations. Leads are also used for sensing purposes, or both sensing and stimulation purposes. The leads are typically coupled to a pacemaker which may be implanted, or may be external to the patient. The pacemaker performs the various sensing and pulsing functions, by receiving and delivering signals through the leads.

Leads may be placed in one or more of a variety of different cardiac locations. In particular, the placement of the leads may be dependent on the cardiac conditions of the patient and the therapy to be delivered. One particular location where leads have been placed is within the interventricular septum that separates the left and right ventricles. For example, one or more leads may be positioned within the interventricular septum by descending the lead into the right atrium, through the right atrial-ventricular valve, and into the right ventricle. The lead may be anchored on the interventricular septum such that it is properly located with respect to the right ventricle. Alternatively, the lead may be pierced into or through the interventricular septum such that it is properly located with respect to the left ventricle. In any event, lead placement within the interventricular septum is of paramount concern because precise placement of the lead can improve the effectiveness of the cardiac pacing therapy delivered to the patient via the lead.

Table 1 below lists a number of documents that disclose various pacemakers and lead configurations.

TABLE 1

| Patent No. | Inventor | Issue Date |
|---|---|---|
| 5,728,140 | Salo et al. | Mar. 17, 1998 |
| 5,683,446 | Gates | Nov. 4, 1997 |
| 5,374,287 | Rubin | Dec. 20, 1994 |
| 3,857,399 | Zacouto | Dec. 31, 1974 |
| 6,263,250 | Skinner | Jul. 17, 2001 |
| 6,078,840 | Stokes | Jun. 20, 2000 |
| 6,049,736 | Stewart et al | Apr. 11, 2000 |
| 5,003,975 | Hafelfinger et al. | Apr. 2, 1991 |
| 4,899,750 | Ekwall | Feb. 13, 1990 |
| 5,534,018 | Wahlstrand et al. | Jul. 9, 1996 |

All patents listed in Table 1 above are hereby incorporated by reference herein in their respective entireties. As those of ordinary skill in the art will appreciate readily upon reading the Summary of the Invention, Detailed Description of the Preferred Embodiments and Claims set forth below, the devices and methods disclosed in the patents of Table 1 may be modified advantageously by using the techniques of the present invention.

### SUMMARY OF THE INVENTION

The present invention has certain objects. That is, various embodiments of the present invention provide solutions to one or more problems existing in the prior art with respect to medical devices in general, and cardiac pacemakers in particular. These problems include, for example, difficulty in precisely positioning a lead within cardiac tissue, and more specifically difficulty in precisely position a lead within the interventricular septum. In particular, conventional implantation techniques may present difficulty in the positioning of a lead within the interventricular septum in close proximity to the left ventricular wall and without piercing the left ventricular wall. Various embodiments of the present invention have the object of solving at least one of the foregoing problems.

It is an object of the invention to improve implantation and positioning of cardiac leads. In particular, it is an object of the invention to facilitate implantation of a lead within the interventricular septum. For example, the lead may include an electrode disposed on the distal end of the lead. Using impedance monitoring techniques outlined in greater detail below, the electrode on the distal end of the lead can be positioned within the interventricular septum without piercing the left ventricular wall. More specifically, the techniques outlined below can facilitate the placement of an electrode disposed on the distal end of the implanted lead precisely within the left ventricular endocardium without piercing the left ventricular wall. In accordance with the invention, impedance measurements give an indication of the precise location of the electrode.

It is a further object of the invention to simplify the procedure associated with the implantation of cardiac leads. By monitoring the impedance between a reference electrode and the electrode disposed on the distal end of a cardiac lead, output can be provided to a surgeon indicative of the placement of the lead within cardiac tissue, such as the interventricular septum. This output can be used by the surgeon to position the distal end of the lead within the interventricular septum without piercing the left ventricular wall. In different embodiments, the output can be generated as visual output such as an impedance curve or numerical representation of impedance, or audible output such as a tone that changes pitch based on the measured impedance. In this manner, the surgical implantation procedure can be simplified and electrode placement accuracy improved.

It is a further object to provide improved lead structures specifically for implantation within cardiac tissue such as the interventricular septum. For example, non-continuous spiral anchoring features are described which can be used to anchor the lead within cardiac tissue. The non-continuous spiral anchoring features may or may not also include fibrous growth structures to further improve fixation of a lead to tissue. In either case, the lead can be used to implant and anchor the electrode within the cardiac tissue using the impedance monitoring techniques in order to ensure that the electrode is properly positioned, e.g., within the interventricular septum without piercing the left ventricular wall.

The invention may offer one or more advantages. For example, the invention may improve and simplify the implantation of cardiac leads. Also, the invention may improve placement accuracy of leads implanted within cardiac tissue, and more specifically within the interventricular septum. In addition, the invention may reduce trauma to a patient by avoiding puncture of the left ventricular wall during the implantation of a lead within the interventricular septum. By avoiding puncture of the left ventricular wall, trauma can be reduced and the likelihood of thrombosis on the electrode disposed on the distal end of the lead can also be reduced.

Various embodiments of the invention may possess one or more features capable of fulfilling the above objects. In general, the invention provides a method of implanting a medical lead that includes measuring impedance associated with an electrode disposed on an implantable lead at a cardiac location, and positioning the electrode based on the measured impedance. By using impedance as an indication of the positioning of the lead, the implantation process can be improved and simplified and negative effects associated with piercing the left ventricular wall can be avoided.

In another embodiment, the invention may be directed to a system comprising an implantable lead including an electrode disposed on the lead. The system may also include an implantation output device coupled to the lead that generates impedance measurements indicative of impedance between the electrode disposed on the lead and a reference electrode, and generates a lead location output signal based on the received impedance measurements. For example, the lead location output signal may be used to generate an audible tone indicative of lead location, a visual output indicative of lead location, or some other output to a surgeon. The surgeon can interpret the audible or visual output as indicating the position of the lead.

In still other embodiments, the invention may be directed to implantable leads or implantable medical devices implementing the implantable leads. In particular, various structures on the implantable leads are described below which can improve the implantation process and improve fixation to cardiac tissue. In addition, an implantable lead that includes an inner lead and an outer lead is described. The inner lead may be threaded within a channel of the outer lead. The outer lead may or may not include an electrode disposed at its distal end. The outer lead can be anchored to the right ventricular wall and the inner lead can be screwed into the interventricular septum as impedance is monitored. The outer lead may be a permanently implantable lead or a more temporary sheath that is used as a tool to implant the inner lead. In the later case, the outer lead may be removed from the patient after the inner lead is implanted in its final position. The inner lead can be advanced to an extent sufficient to precisely position the electrode on the distal end of the inner lead with respect to the left ventricle without piercing the left ventricular wall.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a simplified schematic view of an implantable medical device.

FIG. 2 is a cross-sectional view of a heart having a lead implanted within the interventricular septum in accordance with an embodiment of the invention.

FIG. 3 is a block diagram illustrating components of an implantable medical device in the form of an exemplary cardiac pacemaker.

FIG. 4 is a schematic view of an output device coupled to a lead implanted within a patient during an implantation procedure.

FIG. 5 is a graph of impedance measurements as a function of electrode location within the interventricular septum.

FIG. 6 is a block diagram of an exemplary implementation of an output device coupled to an interventricular electrode to be implanted in the interventricular septum.

FIG. 7 is another graph similar to the graph of FIG. 5, further illustrating a desired location for an electrode of an implantable medical lead within the interventricular septum.

FIG. 8 is an enlarged cross-sectional side-view of an embodiment of a distal end of a lead in accordance with the invention.

FIG. 9 is an enlarged front-view of the lead illustrated in FIG. 8, showing non-continuous spiral anchoring features including fibrous growth structures.

FIG. 10 is an enlarged cross-sectional side-view illustrating the insertion and placement of an outer lead and an inner lead within the interventricular septum.

FIG. 11 is an enlarged cross-sectional side-view illustrating a variation of the lead arrangement shown in FIG. 10.

### DETAILED DESCRIPTION OF THE PREFERRED EMBODIMENTS

In the following detailed description of the preferred embodiments, reference is made to the accompanying drawings that form a part hereof, and in which are shown by way of illustration specific embodiments in which the invention may be practiced. It is to be understood that other embodiments may be utilized and structural or logical changes may be made without departing from the scope of the present invention. The following detailed description, therefore, is not to be taken in a limiting sense, and the scope of the present invention is defined by the appended claims.

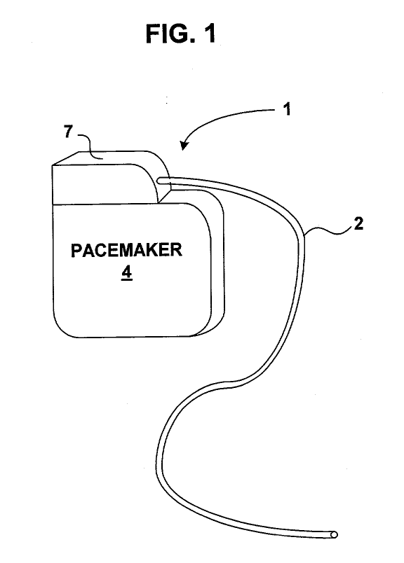

FIG. 1 is a simplified schematic view of an implantable medical device 1 that includes an implantable lead 2. In particular, implantable lead 2 may be implanted within cardiac tissue, such as within the interventricular septum of the heart of a patient, using one or more of the techniques described below. For example, during implantation, impedance measurements can be taken between an electrode disposed on the distal end of lead 2 and a reference electrode. As outlined in greater detail below, measurements of impedance can be used to estimate the location of the distal end of lead 2 within the interventricular septum. In this manner, the distal end of lead 2 can be positioned to a desired location within the interventricular septum of a patient with precision, and without piercing the left ventricular wall.

Lead 2 has a proximal end that can be connected to a pacemaker 4. For example, during normal operation, the proximal end may be connected to a connector module 7, which in turn is coupled to sensing circuitry and/or stimulation circuitry of pacemaker 4. During a lead insertion procedure, however, lead 2 may or may not be coupled to the pacemaker 4. Instead, lead 2 may be coupled to an implantation output device or to both pacemaker 4 and the implantation output device. For example, the implantation output device may process impedance measurements to provide output, such as visual or audio output indicative of the location of a distal end of lead 2 within the interventricular septum of a patient. Energy pulses may be provided at an electrode disposed on the distal end of lead 2 in order to facilitate the impedance measurements.

Lead 2 may be formed from a biocompatible material such as silicone rubber, polyurethane, a silicone-polyurethane copolymer with or without surface modifying end groups. An electrode may be positioned at the distal end of lead 2. In addition, any number of additional electrodes (not shown) may be distributed along the length of lead 2.

The electrode on the distal end of lead 2, as well as other electrodes (if desired) can be made from an electrically conductive, biocompatible material such as elgiloy, platinum, platinum-iridium, platinum-iridium oxide, sintered platinum powder or other residue product after combustion with some high heat source, platinum coated with titanium-nitride, pyrolytic carbon (made via a pyrotechnics manufacturing technique), or the like. In addition, one or more electrodes disposed on lead 2 may function as sensing electrodes to monitor internal electrical signals of the human patient or other mammal in which device 1 is implanted. Although a single lead 2 is shown for purposes of illustration, any number of leads may be used, and thus coupled to connector module 7. In some embodiments, a reference potential may be provided by additional electrodes on lead 2. In other embodiments, the reference potential may be provided not by electrodes carried on lead 2, but by an external reference electrode or a contact surface on an implanted pulse generator, or the like.

The electrode on the distal end of lead 2 may form a substantially cylindrical ring of conductive material that extends about an exterior wall of lead 2. For example, the electrode may extend the entire 360 degrees about lead 2 or some lesser extent. In some embodiments, lead 2 may be tubular but not necessarily cylindrical. For example, lead 2 as well as the electrode disposed on the distal end of lead 2 may have alternative cross sections, e.g., square, rectangular, hexagonal, oval or the like. The electrode may assume a conical shape, with a sharp point to facilitate penetration of the lead in the myocardium. Alternatively, a conical tip may be formed of an electrically non-conducting material. The electrode disposed on the distal end of lead 2 may be coupled to an internal conductor that extends along the length of lead 2. Upon positioning of the distal electrode of lead 2, the proximal end of the lead may be coupled to pacemaker 4, which may be implanted within the patient.

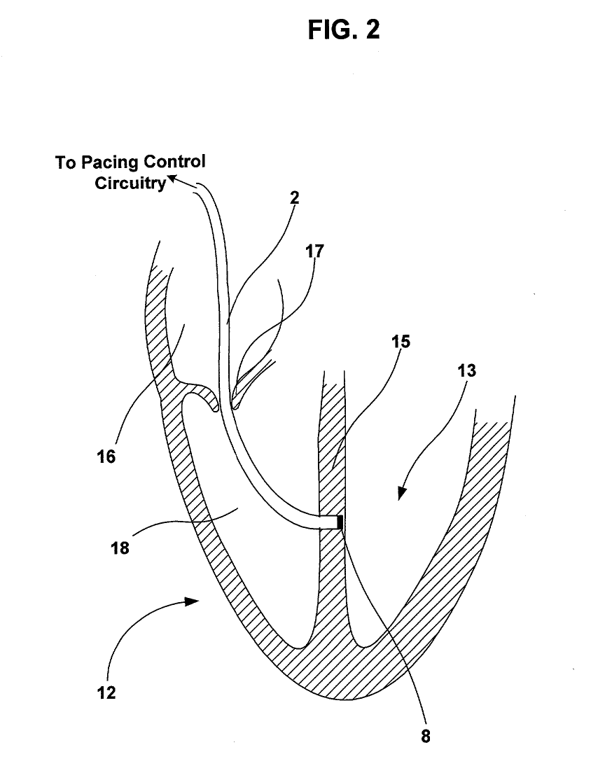

FIG. 2 is a cross sectional view of a heart 12 having lead 2 implanted within the interventricular septum 15. In the example of FIG. 2, a proximal end of lead 2 may be coupled to pacemaker 4 (FIG. 1), which includes pacing control circuitry that performs various cardiac sensing and pacing functions. Electrode 8 disposed on the distal end of lead 2 may be positioned within interventricular septum 15 of heart 12 by descending the lead into right atrium 16, through the right atrial-ventricular valve 17, and into the right ventricle 18. Lead 2 can then pierce the right ventricular wall such that electrode 8 is inserted into the interventricular septum 15.

Precise placement of electrode 8 within interventricular septum 15 is of paramount concern. In particular, precise placement of electrode 8 can improve the effectiveness of the cardiac pacing therapy delivered to the patient via electrode 8 disposed on lead 2. For sensing and stimulation of left ventricle 13, it is highly desirable to position electrode 8 within interventricular septum 15 in close proximity to the left ventricular wall, without piercing the left ventricular wall. For example, it may be especially advantageous to position electrode 8 within the left ventricular endocardium. Such electrode placement can be used to provide pacing therapy effective in achieving optimal hemodynamics.

As outlined in greater detail below, measurements of impedance can be made and used during the implantation procedure to facilitate precision placement of the distal end of lead 2 within the interventricular septum 15, and more specifically within the left ventricular endocardium. Moreover, impedance measurements can be used to ensure that the distal end of lead 2 does not pierce the left-ventricular wall. Various other embodiments of lead structures and signal processing techniques associated with the impedance measurements are also described below.

The implantable medical device may comprise a pacemaker including any number of pacing and sensing leads 2 (one lead shown) attached to a connector module of a hermetically sealed housing (like that shown in FIG. 1) and implanted within a human or mammalian patient. The pacing and sensing lead 2 and possibly other leads sense electrical signals attendant to the depolarization and repolarization of the heart 12, and further provide pacing pulses for causing depolarization of cardiac tissue in the vicinity of the distal end of lead 2. Pacing and sensing lead 2 may have unipolar or bipolar electrodes disposed thereon, as is well known in the art. Examples of a pacemaker include implantable cardiac pacemakers disclosed in U.S. Pat. No. 5,158,078 to Bennett et al., U.S. Pat. No. 5,312,453 to Shelton et al., or U.S. Pat. No. 5,144,949 to Olson, all hereby incorporated by reference herein, each in its respective entirety.

As shown in FIG. 2, pace/sense electrode 8 may sense electrical signals attendant to the depolarization and repolarization of left ventricle 13 heart 12. The electrical signals are conducted to a pacemaker 4 via lead 2. Pace/sense electrode 8 may also deliver pacing pulses for causing depolarization of cardiac tissue in the vicinity of left ventricle 13. The pacing pulses are generated by the pacemaker 4 and are transmitted to pace/sense electrode 8 via lead 2. By using the implantation techniques described in greater detail below, electrode 8 disposed on distal end of lead 2 can be positioned with a level of precision that can improve the therapeutic effect of the implantable medical device. The implantation techniques described below may also simplify the implantation procedure. Structural designs of lead 2 are also described, which can further simplify the implantation procedure and improve placement and fixation of lead 2 within the interventricular septum 15.

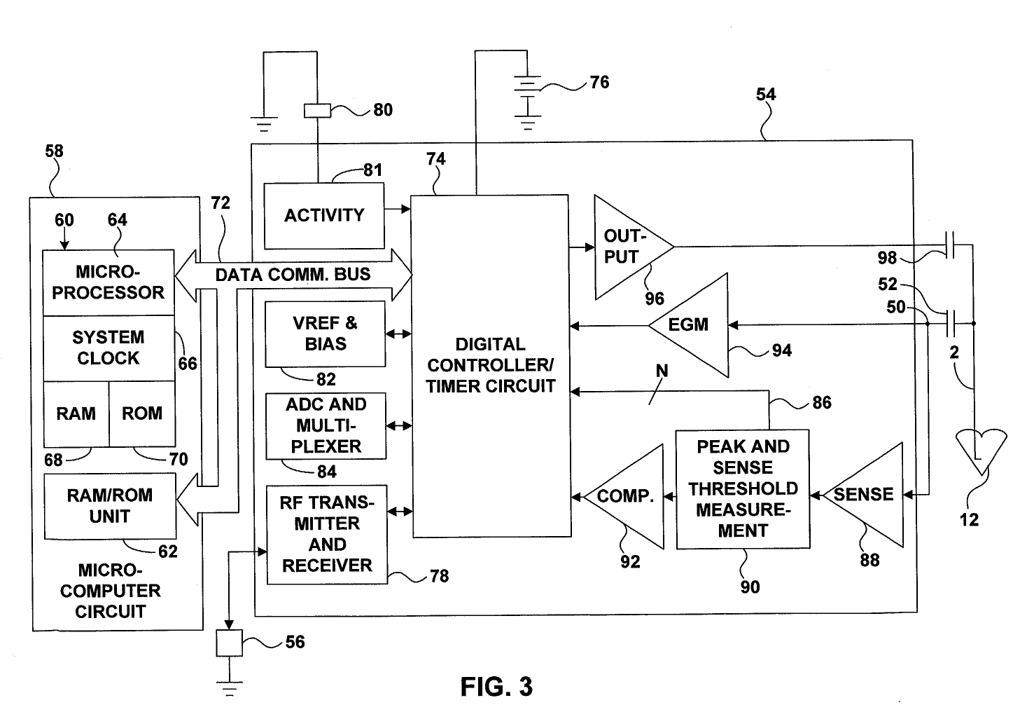

FIG. 3 is a block diagram illustrating the constituent components of an implantable medial device that may make use of a lead 2 in accordance with the present invention. The block diagram in FIG. 3 may correspond to implantable medical device 1 illustrated in FIG. 1. In particular, the device in FIG. 3 may include a pacemaker having a microprocessor-based architecture. The device is shown as including activity sensor or accelerometer 80, which is preferably a piezoceramic accelerometer bonded to a hybrid circuit located inside a housing (such as a housing of pacemaker 4 illustrated in FIG. 1). Activity sensor 80 typically (although not necessarily) provides a sensor output to activity circuitry 81 that varies as a function of a measured parameter relating to a patient's metabolic requirements. Activity circuitry 81 may condition the signal, such as by filtering or analog-to-digital conversion, before forwarding the signal to digital controller 74. For the sake of convenience, the device in FIG. 3 is shown with lead 2 only connected thereto. However, it is understood that similar circuitry and connections not explicitly shown in FIG. 3 apply to any number of additional leads.

The device in FIG. 3 is most preferably programmable by means of an external programming unit (not shown in the figures). One such programmer is the commercially available Medtronic Model 9790 programmer, which is microprocessor-based and provides a series of encoded signals, typically through a programming head which transmits or telemeters radio-frequency (RF) encoded signals. Such a telemetry system is described in U.S. Pat. No. 5,312,453 to Wyborny et al., hereby incorporated by reference herein in its entirety. The programming methodology disclosed in Wyborny et al.'s '453 patent is identified herein for illustrative purposes only. Any of a number of suitable programming and telemetry methodologies known in the art may be employed so long as the desired information is transmitted to and from the pacemaker. In this manner, the implantable medical device in FIG. 3 can be programmed to perform one or more of the pacing and sensing techniques known in the art.

As shown in FIG. 3, lead 2 is coupled to node 50 through input capacitor 52. Activity sensor or accelerometer 80 is most preferably attached to a hybrid circuit located inside hermetically sealed housing 42, e.g., the housing of pacemaker 4 (FIG. 1). The output signal provided by activity sensor 80 is coupled to input/output circuit 54. Input/output circuit 54 contains analog circuits for interfacing with heart 12, activity sensor 80, antenna 56 and circuits for the application of stimulating pulses to heart 12. The rate of heart 12 may be controlled by software-implemented algorithms stored within microcomputer circuit 58.

Microcomputer circuit 58 preferably comprises on-board circuit 60 and off-board circuit 62. Circuit 58 may correspond to a microcomputer circuit disclosed in U.S. Pat. No. 5,312,453 to Shelton et al., hereby incorporated by reference herein in its entirety. On-board circuit 60 preferably includes microprocessor 64, system clock circuit 66, on-board random access memory (RAM) 68 and read-only memory (ROM) 70. Off-board circuit 62 preferably comprises a RAM/ROM unit. On-board circuit 60 and off-board circuit 62 are each coupled by data communication bus 72 to digital controller/timer circuit 74. Microcomputer circuit 58 may comprise a custom integrated circuit device augmented by standard RAM/ROM components. In still other embodiments, the invention may be directed to an implantable medical device comprising one or more implantable leads that include electrodes and a pacemaker coupled to the electrodes via the leads. For example, the pacemaker may include to some or all the components of FIG. 3.

The electrical components shown in FIG. 3 are powered by an appropriate implantable battery power source 76 in accordance with common practice in the art. For the sake of clarity, the coupling of battery power to the various components is not shown in the Figures.

Antenna 56 is connected to input/output circuit 54 to permit uplink/downlink telemetry through RF transmitter and receiver telemetry unit 78. By way of example, telemetry unit 78 may correspond to that disclosed in U.S. Pat. No. 4,566,063 issued to Thompson et al., hereby incorporated by reference herein in its entirety, or to that disclosed in the above-referenced '453 patent to Wybomy et al. It is generally preferred that the selected programming and telemetry scheme permit the entry and storage of cardiac rate-response parameters. The specific embodiments of antenna 56, input/output circuit 54 and telemetry unit 78 presented herein are shown for illustrative purposes only, and are not intended to limit the scope of the present invention.

Continuing to refer to FIG. 3, VREF and Bias circuit 82 most preferably generates stable voltage reference and bias currents for analog circuits included in input/output circuit 54. Analog-to-digital converter (ADC) and multiplexer unit 84 digitizes analog signals and voltages to provide “real-time” telemetry intracardiac signals and battery end-of-life (EOL) replacement functions. Operating commands for controlling the timing of signals generated by the device of FIG. 3 are coupled from microprocessor 64 via data bus 72 to digital controller/timer circuit 74, where digital timers and counters establish the overall escape interval as well as various refractory, blanking and other timing windows for controlling the operation of peripheral components disposed within input/output circuit 54.

Digital controller/timer circuit 74 is preferably coupled to sensing circuitry, including sense amplifier 88, peak sense and threshold measurement unit 90 and comparator/threshold detector 92. Circuit 74 is further preferably coupled to electrogram (EGM) amplifier 94 for receiving amplified and processed signals sensed by lead 2. Sense amplifier 88 amplifies sensed electrical cardiac signals and provides an amplified signal to peak sense and threshold measurement circuitry 90, which in turn provides an indication of peak sensed voltages and measured sense amplifier threshold voltages on multiple conductor signal path 86 to digital controller/timer circuit 74. An amplified sense amplifier signal is also provided to comparator/threshold detector 92. By way of example, sense amplifier 88 may correspond to that disclosed in U.S. Pat. No. 4,379,459 to Stein, hereby incorporated by reference herein in its entirety. Further, digital controller/timer circuit 74 can be programmed to execute various pacing techniques known in the art.

The electrogram signal provided by EGM amplifier 94 is employed when the device is being interrogated by an external programmer to transmit a representation of a cardiac analog electrogram. See, for example, U.S. Pat. No. 4,556,063 to Thompson et al., hereby incorporated by reference herein in its entirety. Output pulse generator 96 provides amplified pacing stimuli to patient's heart 12 through coupling capacitor 98 in response to a pacing trigger signal provided by digital controller/timer circuit 74 each time either (a) the escape interval times out, (b) an externally transmitted pacing command is received, or (c) in response to other stored commands as is well known in the pacing art. By way of example, output amplifier 96 may correspond generally to an output amplifier disclosed in U.S. Pat. No. 4,476,868 to Thompson, hereby incorporated by reference herein in its entirety.

The specific embodiments of sense amplifier 88, output pulse generator 96 and EGM amplifier 94 identified herein are presented for illustrative purposes only, and are not intended to be limiting in respect of the scope of the present invention. The specific embodiments of such circuits may not be critical to practicing embodiments of the present invention so long as they provide means for generating a stimulating pulse and are capable of providing signals indicative of natural or stimulated contractions of heart 12.

In some embodiments of the present invention, the implantable medical device may operate in various non-rate-responsive modes. In other embodiments of the present invention, the implantable medical device may operate in various rate-responsive modes. Some embodiments of the present invention may be capable of operating in both non-rate-responsive and rate-responsive modes. Moreover, in various embodiments of the present invention the implantable medical device may be programmably configured to operate so that it varies the rate at which it delivers stimulating pulses to heart 12 in response to one or more selected sensor outputs being generated. Numerous pacemaker features and functions not explicitly mentioned herein may be incorporated into the implantable medical device while remaining within the scope of the present invention

The present invention is not limited in scope to any particular number of leads or sensors, and is not limited to pacemakers comprising activity or pressure sensors only. In other words, at least some embodiments of the present invention may be applied equally well in the contexts of single-, dual-, triple- or quadruple-chamber pacemakers or other types of pacemakers. See, for example, U.S. Pat. No. 5,800,465 to Thompson et al., hereby incorporated by reference herein in its entirety, as are all U.S. Patents referenced therein. In each case, at least one of the leads may be positioned within the interventricular septum as outlined below.

The implantable medical device illustrated in FIG. 3 may also be a pacemaker combined with a cardioverter and/or defibrillator. Various embodiments of the present invention may be practiced in conjunction with a pacemaker-cardioverter-defibrillator such as those disclosed in U.S. Pat. No. 5,545,186 to Olson et al., U.S. Pat. No. 5,354,316 to Keimel, U.S. Pat. No. 5,314,430 to Bardy, U.S. Pat. No. 5,131,388 to Pless, and U.S. Pat. No. 4,821,723 to Baker et al., all hereby incorporated by reference herein, each in its respective entirety.

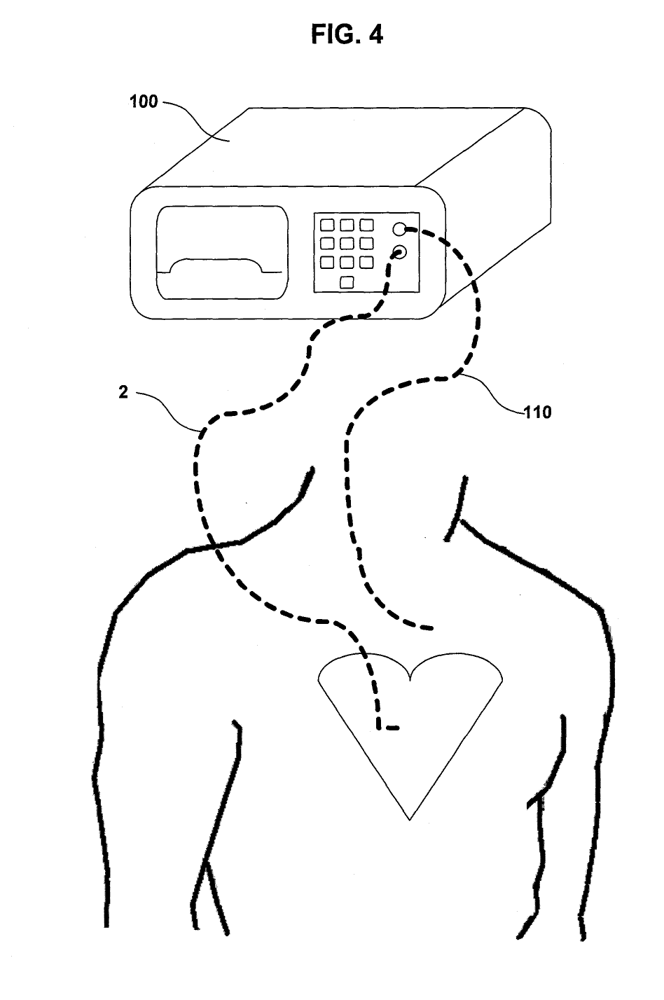

FIG. 4 is a schematic view of an output device 100 coupled to a lead 2 implanted within a patient during an implantation procedure. Also depicted is a lead 110 having a reference electrode. Lead 2 may be implanted within the interventricular septum of a patient's heart by descending the lead into the right atrium, through the right atrial-ventricular valve, and into the right ventricle as shown in FIG. 2. Lead 2 can then be inserted into the interventricular septum of the patient by piercing the distal end of lead 2 through the right ventricular wall. In accordance with the invention, the impedance between the electrode disposed on the distal end of lead 2 and a reference electrode, such as an electrode disposed on the distal end of lead 110, can be measured and used to facilitate precision placement of lead 2 within the interventricular septum.

For example, one or more baseline measurements of impedance can be made when the electrode is in the left ventricle. Then, as the electrode pierces the interventricular septum and advances toward the left ventricle, additional impedance measurements can be taken. These impedance measurements can be used to determine the position of the electrode in relation to the left ventricular wall. Accordingly, using the impedance measurements, the electrode can be advanced to close proximity to the left ventricular wall without piercing the wall.

Implantation output device 100 may generate one or more signals indicative of the impedance between the electrode disposed on the distal end of lead 2 and the reference electrode. The reference electrode is generally stationary, and may be an external electrode affixed to the patient's body. Thus, measured changes in impedance may correspond to movement of the electrode disposed on the distal end of lead 2 in relation to the interventricular septum. For example, the impedance measured when the electrode disposed on the distal end of lead 2 is located within a heart chamber may be approximately one-third (⅓) of the impedance measured when the same electrode is positioned within tissue associated with the interventricular septum. This difference may result from the fact that the impedance of blood is approximately one-third the impedance of cardiac tissue. The invention recognizes and exploits the relationship between impedance and the location of the electrode on the distal end of lead 2 in order to simplify and improve the implantation of lead 2 within the interventricular septum.

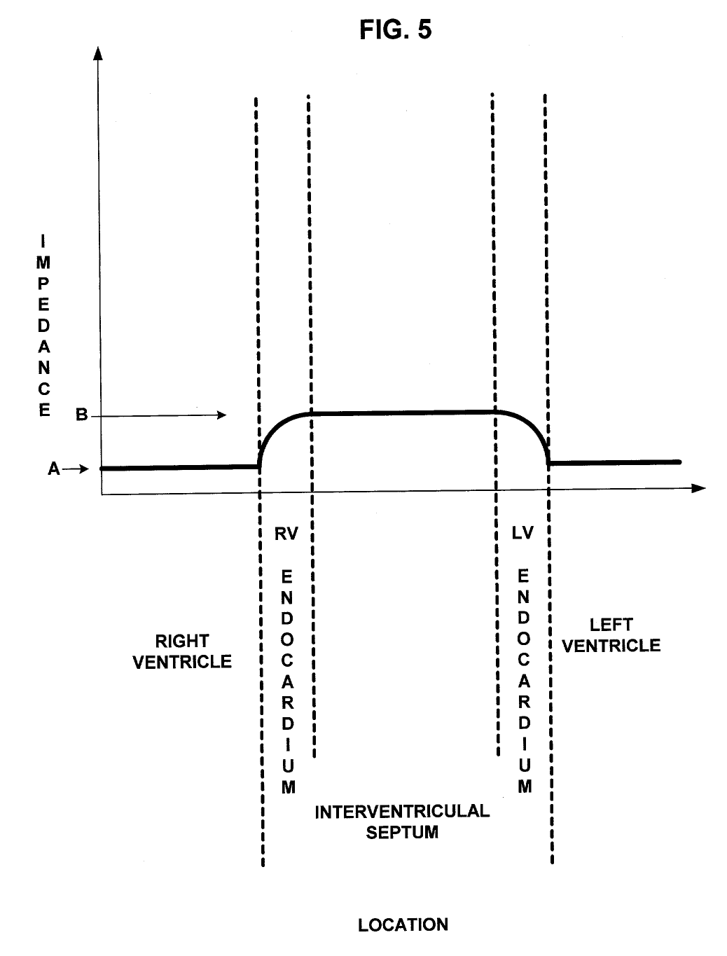

FIG. 5 is a graph of impedance measurements as a function of electrode location within the interventricular septum. As shown, impedance has a generally constant value (A) when measured between the stationary reference electrode and an electrode positioned in the right ventricle. One or more baseline measurements can be taken when the electrode is in the right ventricle to define value (A). As the electrode moves from the right ventricle, piercing the right ventricular wall and entering the right ventricle (RV) endocardium, impedance generally increases in a non-linear fashion. Impedance generally plateaus near a constant value (B) when the electrode is positioned within a central region of the interventricular septum. Value (B) is typically larger than value (A). For example, value (B) may be approximately 3*value (A).

As further depicted in FIG. 5, impedance generally decreases in a non-linear fashion as the electrode moves into the left vertical (RV) endocardium. In other words, after plateauing near value (B), impedance decreases in a non-linear fashion as the electrode approaches the left ventricular wall. If the electrode pierces the left ventricular wall, impedance generally returns to the constant value (A).

In accordance with the invention, this impedance curve illustrated in FIG. 5 can be exploited to provide an indication of the position of the electrode disposed on the distal end of lead 2 within the interventricular septum. In particular, by tracking impedance as the lead 2 begins in the right ventricle, pierces the interventricular septum and approaches the left ventricular wall, the electrode disposed on the distal end of the lead can be precisely positioned within the LV endocardium in close proximity to the left ventricular wall, without piercing the left ventricular wall. Such precision implantation can improve pacemaker therapy, reduce patient trauma, and reduce the likelihood of negative effects such as thrombosis occurring on the positioned electrode.

In particular, it is highly desirable to avoid piercing the left ventricular wall. If the left ventricular wall is pierced, the patient may experience more trauma. Moreover, if the left ventricular wall is pierced, thrombus may be more likely to occur on the electrode disposed on the distal end of lead 2 because the electrode is exposed to blood flow in the left ventricle. Further, if a thrombus develops and then embolizes, entering the blood stream, more serious problems such as stroke and possibly death can result. For these reasons, it is highly desirable to avoid piercing the left ventricular wall. Still, improved therapeutic value may be achieved by positioning the electrode in very close proximity to the left ventricular wall, albeit without piercing the wall.

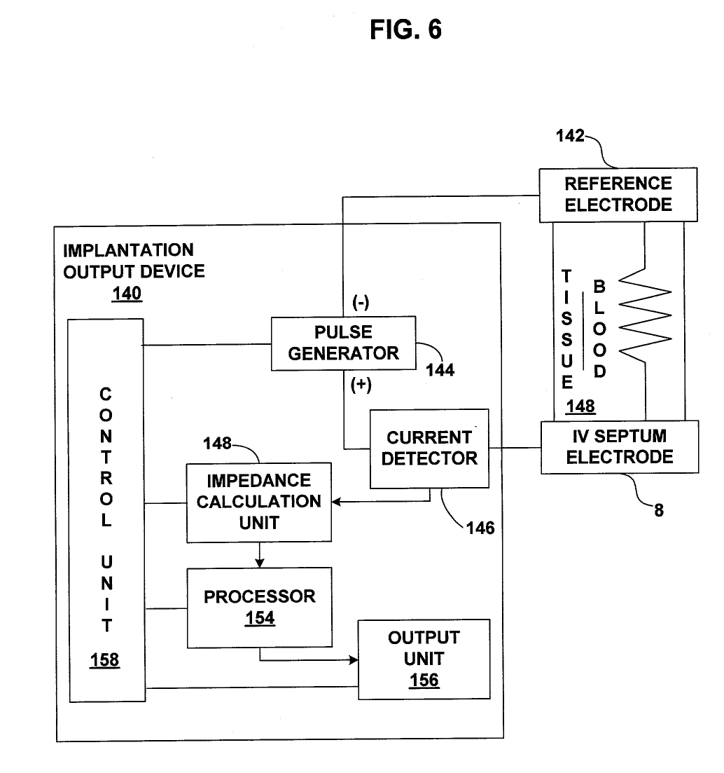

FIG. 6 is a block diagram of an exemplary implementation of an implantation output device 140 coupled to a reference electrode 142 and an electrode 8 to be implanted in the interventricular septum. Implantation output device 140 may correspond to output device 100 (FIG. 4), or may be a different device. As shown in FIG. 6, implantation output device 140 may be coupled to the proximal end of lead 2 during the implantation procedure during which lead 2 positions electrode 8 within the interventricular septum. The reference electrode may be coupled to lead 2, a separate lead as illustrated in FIG. 4, or even some other grounded structure that can provide a reference potential.

As shown in FIG. 6, implantation output device 140 includes a pulse generator 144 such as a voltage source, that provides pulses to electrode 8 during the implantation procedure. For example, the pulses provided to electrode 8 during the implantation procedure may comprise periodic pulses, or a more continuous stream of pulses. In one example, the pulses comprise a high frequency lead integrity signal. In each case, current detector 146 can measure the current that flows through the circuit formed by pulse generator 144, electrode 8, the blood and/or tissue of the patient 148, and a grounded reference electrode 142. The pulses provided to facilitate impedance measurements may comprise sub- or supra-threshold pacing pulses with amplitude between 0.1 and 5 volts and with pulse widths in the range that can be programmed, e.g., 0.05 to 1.5 ms. Alternatively, continuous alternating current may be applied to facilitate the impedance measurements using sine wave or rectangular block shaped alternating current waveforms. The continuous alternating current may be in the form of a cyclic current with a duty cycle between 10 and 50 percent and a repetition rate between 0.5 Hz and 5 Hz.

Given the magnitude of a voltage signal generated by pulse generator 144 and an estimation of current provided by current generator 146, impedance calculation unit 148 can calculate an impedance value associated with the location of electrode 8. For example, as electrode 8 is inserted into the interventricular septum as outlined above, the impedance between electrode 8 and reference electrode may change, such as illustrated in the graph of FIG. 5. Impedance calculation unit 148 can exploit the relationship of Ohm's law (V=I*Z, or voltage=current*impedance) in order to calculate the impedance or the change in impedance associated with the positioning of electrode 8.

A processor 154 may receive the calculated impedance values, or the calculated changes in impedance and use the received impedance to generate and provide a lead location output signal to output unit 156. Control unit 158 may be coupled to some or all of the components of implantation output device to control the signal generation, impedance estimation and output provided by implantation output device 140.

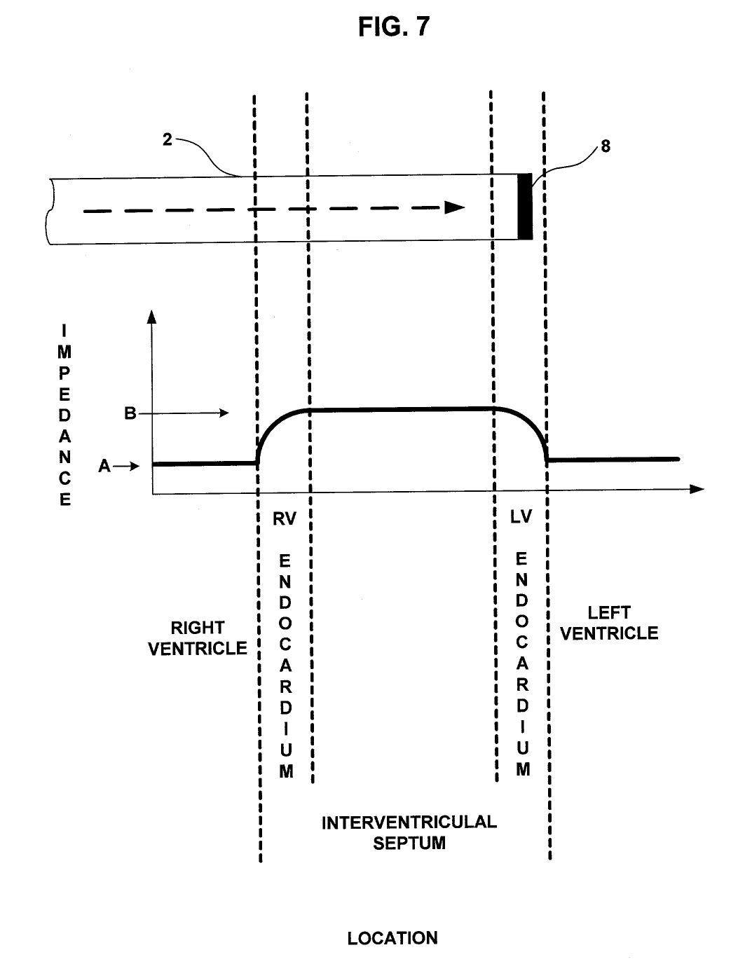

Output unit 156 may correspond to a video display that displays the impedance curve illustrated in FIG. 5. For example, the video display may comprise a liquid crystal display (LCD), a cathode ray tube (CRT), a plasma display, or the like. In any case, processor 154 can receive the calculated impedance values or calculated changes in impedance and use the impedance to generate a lead location output signal that drives the video display to present the impedance curve. Additionally, the video display may also superimpose on the impedance curve a representation of lead 2 in relation to the interventricular septum (such as a video representation of FIG. 7 discussed below).

Alternatively or additionally, output unit may include a speaker to provide the surgeon with an audible indication of the location of lead within the interventricular septum. In that case, processor 154 can receive the calculated impedance values or calculated changes in impedance and use the impedance to generate a lead location output signal that drives the speaker in a manner that relates impedance to speaker output. For example, an audible tone may be created as a function of the measured impedance. The tone may increase or decrease in pitch or volume as a function of the measured impedance. Thus, a surgeon may hear an audible tone that increases in pitch as electrode 8 enters the RV endocardium, plateaus at a high pitch when electrode 8 is in the center portion of the interventricular septum, and begins to decrease in pitch as electrode 8 enters the LV endocardium and approaches the left ventricular wall.

By using the output provided by output unit 156, the surgeon can precisely position electrode 8 on the distal end of lead 2 to a desired location within the interventricular septum without piercing the left ventricular wall. In other embodiments, output unit 156 may simply provide a visual indication or numerical representation of the measured value of either impedance or a change in impedance, which the surgeon can interpret as he or she deems appropriate. Importantly, using impedance measurements associated with the location of electrode 8 within the interventricular septum can improve the precision of the implantation process. With added precision, patient trauma can be reduced and thrombosis or other negative effects associated with piercing of the left ventricular wall can be avoided.

FIG. 7 is another graph similar to that in FIG. 5, but further illustrating one desired location for an electrode of an implantable medical lead within the interventricular septum. As mentioned above, such a graphical representation of impedance as shown in FIG. 7 as well as a representation of lead 2 in relation to the interventricular septum may be provided by output unit 156 of implantation output device 140 for presentation to a surgeon during the implantation procedure.

As shown in FIG. 7, measured impedance may be a direct function of the location of electrode 8 on the distal end of lead 2 in relation to the interventricular septum. Again, impedance has a generally constant value (A) when measured between the stationary reference electrode and an electrode 8 positioned in the right ventricle. As the electrode 8 moves from the right ventricle, piercing the right ventricle (RV) endocardium, impedance generally increases in a non-linear fashion. Impedance generally plateaus near a constant value (B) when the electrode 8 is positioned within a center region of the interventricular septum. Impedance generally decreases in a non-linear fashion as the electrode moves into the left vertical (LV) endocardium. Then, if the electrode pierces the left ventricular wall, impedance generally returns to the constant value (A). Accordingly, it is desirable to insert lead 2 within the interventricular septum past the point where impedance plateaus near a constant value (B) but before the point where impedance returns to the constant value (A). In this manner, placement of electrode 8 in close proximity to the left ventricular wall without piercing the wall can be achieved. Such precise placement of electrode 8 can ensure that maximal hemodynamics are obtained. In other words, such precise placement of electrode 8 can improve the therapy to be provided to the patient via the electrode.

The measured impedance may substantially correspond to a resistive component of impedance. Thus, the techniques may be applied using resistance measurements, which in most cases should be the same as impedance measurements.

Techniques for advancing lead 2 within the interventricular septum can be further improved and/or simplified by using one or more lead structures outlined in greater detail below. The lead structures outlined below may or may not be used in conjunction with the impedance monitoring techniques outlined above. When the structures outlined below are used in combination with the impedance monitoring techniques the overall precision and/or simplicity of the implantation procedure may be improved.

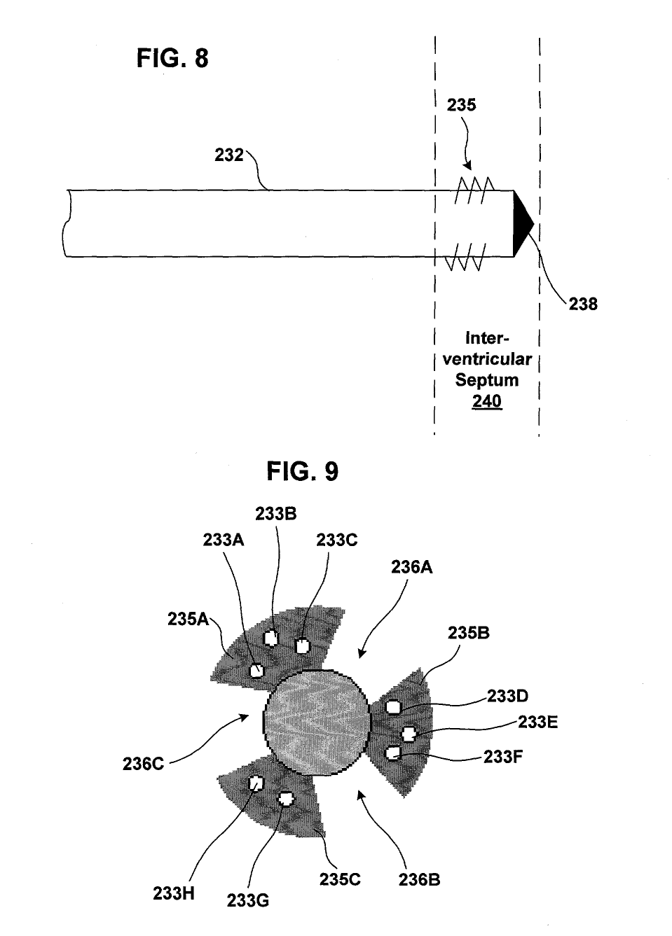

FIG. 8 is an enlarged cross-sectional side-view of an embodiment of a distal end of a lead 232 in accordance with the invention. For example, lead 232 may correspond to lead 2 outlined above. In particular, lead 232 may be used in the implantation procedure that uses impedance measurements to estimate and pinpoint lead location within the interventricular septum.

In the example of FIG. 8, lead 232 incorporates spiral structures in the form of non-continuous spiral anchoring features 235. The non-continuous spiral anchoring features 235 may or may not be formed with fibrous growth structures to enhance fixation to tissue within interventricular septum 240. The growth structures may take the form of holes in the anchoring features 235. The non-continuous spiral anchoring features 235 may comprise a segmented threading that can be used to screw lead 232 into interventricular septum 240 and thereby fix electrode 238 with respect to a desired location. The electrode 238 may assume a conical shape, with a sharp point to facilitate penetration of the lead in the myocardium. Alternatively, the conical tip may be formed of an electrically non-conducting material with an electrode disposed adjacent the conical tip.

The non-continuous nature of non-continuous spiral anchoring features 235 can enhance fibrous tissue growth around and between the non-continuous spiral anchoring features 235 and prevent rotation of the lead around its longitudinal axis to improve fixation of lead 232 within interventricular septum 240. Fibrous growth holes formed in the non-continuous spiral anchoring features 235 may also improve fixation. A continuous spiral structured lead, with or without fibrous growth features could also be used.

Each of the individual spiraled anchoring features (also referred to as spiraled segments) that make up the non-continuous spiral anchoring features may be made from a metal (coated or not coated with an electrically insulating material) or sufficiently rigid polymer material, such as polyurethane or silicone rubber. The size and depths of the individual segments may extend approximately 0.5-2.0 mm outward from lead body. Thus, the diameter of the non-continuous spiral anchoring features may be approximately 2*(0.5-2 mm) larger than lead body diameter. By way of example, the pitch of the spiral segments may range from about 1 to 3 mm, although the invention is not necessarily limited in that respect.

The non-continuous spiral anchoring features can be formed in an injection mold and may be made from a non-corrosive steel alloy. The holes in each of the segments may be formed by corresponding cylindrical pins that are placed in the mold prior to injection, and then removed from the mold prior to the opening of the mold, or during the opening of the mold.

FIG. 9 is a close-up front-view of non-continuous spiral anchoring features 235A-235C (collectively non-continuous spiral anchoring features 235) formed with fibrous growth holes 233A-233H (collectively fibrous growth holes 233). Non-continuous spiral anchoring features 235 are spiraled with respect to one another, and intermittent open spaces 236A-236C (collectively gaps 236) separate respective non-continuous spiral anchoring features 235. The non-continuous spiral anchoring features 235 as well as fibrous growth holes 233 improve fixation of lead 232 to tissue within interventricular septum 240.

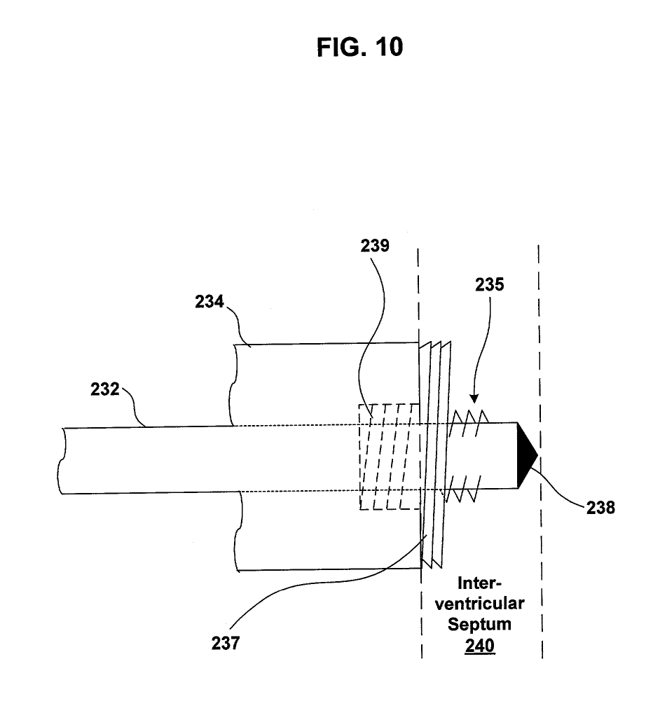

FIG. 10 is a close-up cross-sectional side-view illustrating the placement of the lead illustrated in FIGS. 8 and 9 within the interventricular septum. The structure in FIG. 10 includes an outer lead 234 and an inner lead 232. Both outer lead 234 and inner lead 232 include a proximal end and a distal end. Inner lead 232 is originally surrounded by outer lead 234, which can be used to guide the inner lead 232 to the desired location within the patient. In this case, outer lead 234 guides the lead 232 to the interventricular wall. Outer lead 234 includes a threaded channel 239 and the non-continuous spiral anchoring features of the inner lead 232 can threadedly engage the threaded channel 239 of the outer lead 234. Outer lead 234 may be a permanently implantable lead, or alternatively may comprise a temporary sheath that is used as a tool to implant the inner lead 232. In the later case, outer lead 234 may be removed from the patient after the inner lead 232 is implanted in its final position.

Outer lead 234 also includes anchoring features 237 such as a single filar or multifilar helix, or screw-like spiraled grooves that can be used to fix the outer lead 234 to the interventricular wall. Then, inner lead 232 can be screwed into the interventricular septum 240 to a desired location with respect to the left ventricle. In particular, one or more of the impedance measuring techniques described above can be used to ensure that inner lead 232 is positioned properly within the interventricular septum 240 without piercing the left ventricular wall. In other words, as lead 232 is screwed into the interventricular septum 240, the impedance measurements can be used to pinpoint the location of the lead. When lead 232 reaches the desired location, as determined by the impedance, screwing can be discontinued. At that point, the spiral anchoring features of inner lead, e.g., non-continuous spiral anchoring features 235 formed with fibrous growth holes 233 may improve fixation of lead 232 to tissue within interventricular septum 240. Again, the electrode 238 may assume a conical shape, with a sharp point to facilitate penetration of the lead in the myocardium. Alternatively, the conical tip may be formed of an electrically non-conducting material with an electrode disposed adjacent the conical tip.

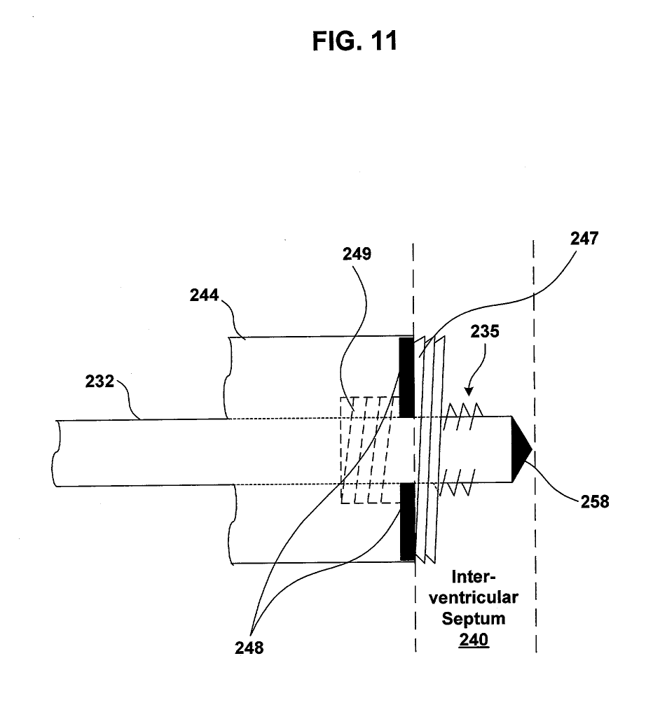

FIG. 11 is an enlarged cross-sectional side-view illustrating a variation of the embodiment of FIG. 10 in which the outer lead includes an electrode for sensing or stimulating the right ventricle. Again, both outer lead 244 and inner lead 232 include a proximal end and a distal end. Inner lead 232 is originally surrounded by outer lead 234, which can be used to guide the inner lead 232 to the desired location within the patient. In this case, outer lead 244 guides inner lead 232 to the interventricular wall. Outer lead 244 includes a threaded channel 249 and the non-continuous spiral anchoring features 235 of inner lead 232 can threadedly engage the threaded channel.

Outer lead 244 also includes anchoring features 247 such as a single filar or multifilar helix, or screw-like spiraled grooves that can be used to fix the outer lead 244 to the interventricular wall. In addition outer lead 244 further includes an electrode 248 disposed in close proximity to the distal end, wherein the anchoring features 247 of the outer lead are disposed adjacent the electrode 248 of the outer lead.

Electrode 248 of outer lead 244 can be a reference electrode used in the impedance measuring techniques described above. Additionally or alternatively, electrode 248 of outer lead 244 may be a simulating and/or sensing electrode used for simulating and/or sensing the right ventricle. In either case, once outer lead 244 is anchored to the right ventricular wall such that electrode 248 is in physical contact with the right ventricular wall. Then, inner lead 232 can be screwed into the interventricular septum 240 to a desired location with respect to the left ventricle. In particular, one or more of the impedance measuring techniques described above can be used to ensure that inner lead 232 is positioned properly within the interventricular septum 235 without piercing the left ventricular wall. The spiral anchoring features of inner lead, e.g., non-continuous spiral anchoring features 235 formed with fibrous growth holes 233 can improve fixation of lead 232 to tissue within interventricular septum 240 once it is properly positioned using the impedance measurements. The inner electrode 258 may assume a conical shape, with a sharp point to facilitate penetration of the lead in the myocardium. Alternatively, the conical tip may be formed of an electrically non-conducting material with an inner electrode disposed adjacent the conical tip.

The preceding specific embodiments are illustrative of the practice of the invention. It is to be understood, however, that in light of this disclosure, other embodiments will become apparent to those skilled in the art. For example, some embodiments may be practiced in an external (non-implantable) or a partially external medical devices. Also, the impedance measuring and monitoring techniques may be used to improve lead placement in other cardiac tissue locations. In other words, the impedance measuring and monitoring techniques used to place a lead within the interventricular septum are one exemplary embodiment of the broader concept of using impedance measuring and monitoring to facilitate precise lead placement within cardiac tissue, or possibly even other non-cardiac cellular tissue. Accordingly, these and other embodiments are within the scope of the following claims.

In the claims, means-plus-function clauses are intended to cover the structures described herein as performing the recited function and not only structural equivalents, but also equivalent structures. Thus, although a nail and a screw may not be structural equivalents in that a nail employs a cylindrical surface to secure wooden parts together, whereas a screw employs a helical surface, in the environment of fastening wooden parts, a nail and a screw are equivalent structures.

1. A method of implanting a medical lead comprising:
measuring electrical impedance between an electrode disposed on an implantable lead and a reference potential as the electrode is positioned within cardiac tissue ; and
positioning the electrode at a location in the cardiac tissue based on the measured impedance.

2. The method of claim 1, wherein the electrode is a conical shaped electrode disposed at a distal end of the lead.

3. The method of claim 1, wherein measuring impedance comprises measuring resistance.

4. The method of claim 1, further comprising:
inserting the lead into an interventricular septum;
measuring impedance associated with an electrode disposed on a distal end of the lead within an interventricalar septum location; and
positioning the electrode within the interventricular septum based on the measured impedance.

5. The method of claim 4, further comprising positioning the electrode without puncturing a left ventricular wall.

6. The method of claim 4, further comprising positioning the electrode within a left ventricular endocardium based on the measured impedance.

7. The method of claim 1, wherein positioning the electrode based on the measured impedance further includes receiving an audible sound indicative of measured impedance and positioning the electrode based on the audible sound.

8. The method of claim 1, further comprising delivering an electrical signal at the cardiac location;
measuring impedance between the electrode disposed on an implantable lead and a reference electrode in response to the delivered signal; and
positioning the electrode based on the measured impedance.

9. The method of claim 8, further comprising:
inserting the lead into an interventricular septum;
delivering the electrical signal to the heart at an interventricular location associated with an electrode disposed on a distal end of the lead;
measuring impedance between the electrode disposed on a distal end of the lead and the reference electrode; and
positioning the electrode within the interventricular septum based on the measured impedance.

10. The method of claim 9, wherein inserting the lead into an interventricular septum comprises:
anchoring an outer lead to a right ventricular wall; and
inserting an inner lead into the interventricular septum.

11. The method of claim 10, wherein outer lead includes an outer lead electrode positioned adjacent a right ventricular wall upon anchoring an outer lead to a right ventricular wall.

12. The method of claim 11, wherein the outer lead electrode is the reference electrode.

13. The method of claim 11, wherein the outer lead electrode is a stimulation electrode for stimulating a right ventricle.

14. The method of claim 1, wherein the implantable lead includes non-continuous spiral anchoring features, and wherein positioning the electrode based on the measured impedance comprises screwing non-continuous anchoring features of the lead into the cardiac location.

15. The method of claim 14, wherein the non-continuous spiral anchoring features include fibrous growth holes.

16. The method of claim 1, wherein measuring impedance comprises measuring impedance between the electrode disposed on the implantable lead and a reference electrode disposed on a pacemaker.

17. The method of claim 1, wherein measuring impedance comprises measuring impedance between the electrode disposed on the implantable lead and a reference electrode in response to a stimulation pulse provided at the electrode disposed on the implantable lead.

18. The method of claim 1, wherein measuring impedance comprises measuring impedance between the electrode disposed on the implantable lead and a reference electrode in response to a high frequency lead integrity signal provided at the electrode on the implantable lead.

19. A system comprising:
an implantable lead including an electrode disposed on the lead; and
an implantation output device coupled to the lead that generates impedance measurements indicative of impedance between the electrode disposed on the lead and a reference electrode, and generates a lead location output signal based on the impedance measurements.

20. The system of claim 19, lead location output signal provides an indication of lead location within an interventricular septum.

21. The system of claim 19, wherein the implantation output device further includes a display, wherein lead location output signal provides visual output via the display indicative of lead location within an interventricular septum.

22. The system of claim 19, wherein the implantation output device further includes a speaker, wherein lead location output signal provides audio output via the speaker indicative of lead location within an interventricular septum.

23. An implantable medical lead comprising:
an outer lead including a proximal end, a distal end, and anchoring features disposed at the distal end, wherein the outer lead further includes a threaded channel; and
an inner lead including a proximal end, a distal end, and an electrode disposed at the distal end of the inner lead, wherein the inner lead includes spiral anchoring features that threadedly engage the threaded channel.

24. The implantable medical lead of claim 23, wherein the spiral anchoring features comprise non-continuous spiral anchoring features.

25. The implantable medical lead of claim 24, wherein the non-continuous spiral anchoring features include fibrous growth holes.

26. The implantable medical lead of claim 23, wherein the outer lead further includes an electrode disposed in close proximity to the distal end, wherein the anchoring features of the outer lead are disposed adjacent the electrode of the outer lead.

27. An implantable medical device comprising:
a medical lead including:
an outer lead including a proximal end, a distal end, and anchoring features disposed at the distal end, wherein the outer lead further includes a threaded channel; and
an inner lead including a proximal end, a distal end, and an electrode disposed at the distal end of the inner lead, wherein the inner lead includes spiral anchoring features that threadedly engage the threaded channel; and
a pacemaker coupled to the proximal ends.

28. The device of claim 27, wherein the pacemaker controls stimulation pulses delivered by at least one of the electrodes.

29. The device of claim 27, wherein the pacemaker processes signals detected by at least one of the electrodes.

30. The device of claim 27, wherein the spiral anchoring features comprise non-continuous spiral anchoring features.

31. The device of claim 30, wherein the non-continuous spiral anchoring features define fibrous growth holes.

32. The device of claim 27, wherein the outer lead further includes an electrode disposed in close proximity to the distal end, wherein the anchoring features of the outer lead are disposed adjacent the electrode of the outer lead.

33. A system comprising:
an implantable lead including an electrode; and
a lead location means coupled to the lead, for generating impedance measurements indicative of impedance between the electrode disposed on the lead and a reference electrode and generating a lead location output signal based on the received impedance measurements.

34. The system of claim 33, wherein the lead location output signal provides an indication of lead location within an interventricular septum.

35. The system of claim 33, wherein the implantation output device further includes a display, wherein lead location output signal provides visual output via the display indicative of lead location within an interventricular septum.

36. The system of claim 35, wherein the implantation output device further includes a speaker, wherein lead location output signal provides audio output via the speaker indicative of lead location within an interventricular septum.

37. An implantable medical lead comprising:
an outer lead including a proximal end, a distal end, and means for anchoring the outer lead on a right ventricular wall disposed at the distal end, wherein the outer lead further includes a threaded channel; and
an inner lead including a proximal end, a distal end, and an electrode disposed at the distal end of the inner lead, wherein the inner lead includes means for anchoring the inner lead into an interventricular septum, wherein the means for anchoring the inner lead threadedly engages the threaded channel.

38. The implantable medical lead of claim 37, wherein the means for anchoring the inner lead include non-continuous spiral anchoring features.

39. The implantable medical lead of claim 38, wherein the non-continuous spiral anchoring features include fibrous growth holes.

40. The implantable medical lead of claim 37, wherein the outer lead further includes an electrode disposed in close proximity to the distal end, wherein means for anchoring the outer lead is disposed adjacent the electrode of the outer lead.
